(19) United States
(12) Patent Application Publication
(10) Pub. No.: US 2008/0188862 A1
(43) Pub. Date: Aug. 7, 2008

(54) Method of treatment with endoscope

(75) Inventor: Nariaki Saitou, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMOUS MEDICAL SYSTEMS CORP., Tokyo (JP)

(21) Appl. No.: 11/701,349

(22) Filed: Feb. 1, 2007

(51) Int. Cl. A61B 1/00

(52) U.S. Cl. 606/113

(57) ABSTRACT

A method of treatment with an endoscope for treating an affected part to be treated, using the endoscope having an observation optical system and an opening, opened to the distal end of an endo-therapy accessories insertion channel, at a distal end portion of an insertion portion provided with the endo-therapy accessories insertion channel therein, and having an endo-therapy accessories forceps elevator arranged in the opening for elevating endo-therapy accessories in a predetermined direction, the endo-therapy accessories including a loop shape portion projected from the opening at the distal end of a sheath portion, includes: a first step of setting to an observation state to observe the affected part within an observation visual field by the observation optical system and bringing the loop shape portion projected from the opening, in a state that a loop of the loop shape portion is widened, into contact with a surface of the affected part; and a second step of pressing the widened loop shape portion against the surface of the affected part by elevator control for elevating the endo-therapy accessories forceps elevator while maintaining the state where the loop shape portion is in contact with the surface of the affected part, so as to cause the affected part inside the loop shape portion to bulge out.

### BACKGROUND OF THE INVENTION

1. Field of the Invention

The present invention relates to a method of treatment with an endoscope for performing treatment by inserting endo-therapy accessories provided with a loop shape portion in a channel.

2. Description of Related Art

In recent years, an endoscope has become widely used, wherein an insertion portion thereof is inserted in a body cavity, the body cavity is observed by an observation optical system in an observation window provided at a distal end portion thereof, and medical treatment is performed on an affected part, such as a polyp, that needs to be treated using endo-therapy accessories.

When treating the affected part, such as the polyp, the endo-therapy accessories utilizing high-frequency current, called a high-frequency snare, with a loop-shaped wire provided at the distal end of a sheath is often used.

For example, the first prior art document, Japanese Patent Laid-Open No. 2000-342600, describes in the paragraph [0019] that “by pressing the distal end of a sheath 1 against a polyp 100, the polyp 100 bulges out while the distal end of the sheath 1 digs into the root of the polyp 100 and is stabilized.”

The second prior art document, Japanese Patent Laid-Open No. 2001-252280, discloses in the paragraphs [0018] and [0019] that a snare loop 3 is pressed against a mucosal surface 100, thereby a polyp 101 as a whole can be cinched at a boundary portion with the mucosal surface 100 and excised without exception.

The third prior art document, Japanese Patent Laid-Open No. 2005-152452, discloses an endoscope with an endo-therapy accessories forceps elevator arranged in an opening of a channel adjacent to an observation window provided at a distal end portion of an insertion portion.

The endo-therapy accessories forceps elevator is coupled with a elevator control knob provided on a manipulation portion of the endoscope via a wire inserted in the insertion portion, and has a structure where the endo-therapy accessories forceps elevator can rotate to change a projection direction of the distal end side of the endo-therapy accessories projected from the opening by an operator rotating the elevator control knob.

By the operator manipulating the elevator control knob, the projection direction of the distal end side of the endo-therapy accessories with respect to the observation window can be changed in the vertical direction, for example.

Moreover, the fourth prior art document, U.S. Pat. No. 7,063,661 discloses that a part to be excised bulges out by pressing the snare loop against the affected part and subsequently performing suction.

### SUMMARY OF THE INVENTION

A method of treatment with an endoscope according to an embodiment for treating an affected part to be treated, using the endoscope having an observation optical system and an opening, opened to the distal end of an endo-therapy accessories insertion channel, at a distal end portion of an insertion portion provided with the endo-therapy accessories insertion channel therein, and having an endo-therapy accessories forceps elevator arranged in the opening for elevating endo-therapy accessories in a predetermined direction, the endo-therapy accessories including a loop shape portion projected from the opening at the distal end of a sheath portion, includes:

a first step of setting to an observation state to observe the affected part within an observation visual field by the observation optical system and bringing the loop shape portion projected from the opening, in a state that a loop of the loop shape portion is widened, into contact with a surface near the affected part; and

a second step of pressing the widened loop shape portion against the surface near the affected part by elevator control for elevating the endo-therapy accessories forceps elevator while maintaining the state where the loop shape portion is in contact with the surface near the affected part, so as to cause the affected part inside the loop shape portion to bulge out.

### BRIEF DESCRIPTION OF THE DRAWINGS

FIG. 1 shows a configuration of an endoscope apparatus according to a first embodiment of the present invention;

FIG. 2 is a front view of a distal end portion of a direct view type endoscope;

FIG. 3 is an A-A sectional view of FIG. 2;

FIG. 4 is a B-B sectional view of FIG. 2;

FIG. 5 is a flow chart showing an example of procedure of a method of treatment according to the first embodiment;

FIGS. 6A and 6B are explanatory diagrams of Step S1 in FIG. 5;

FIGS. 7A and 7B are explanatory diagrams of Step S2 in FIG. 5;

FIGS. 8A and 8B are explanatory diagrams of Step S3 in FIG. 5;

FIGS. 9A and 9B are explanatory diagrams of Step S4 in FIG. 5;

FIG. 10 is a flow chart showing an example of the procedure of a modified method of treatment;

FIGS. 11A and 11B are explanatory diagrams of Step S11 in FIG. 10;

FIGS. 12A and 12B are explanatory diagrams of Step S12 in FIG. 10;

FIGS. 13A and 13B are explanatory diagrams of Step S13 in FIG. 10;

FIGS. 14A and 14B are explanatory diagrams of Step S14 in FIG. 10;

FIG. 15 is a front view of the distal end portion of the endoscope according to a modification;

FIG. 16 is a C-C sectional view of FIG. 15;

FIG. 17 is a front view of the distal end portion of an oblique view type endoscope according to a second embodiment of the present invention;

FIG. 18 is a D-D sectional view of FIG. 17;

FIG. 19 is a flow chart showing an example of the procedure of the method of treatment according to the second embodiment;

FIGS. 20A and 20B are explanatory diagrams of Step S21 in FIG. 19;

FIGS. 21A and 21B are explanatory diagrams of Step S22 in FIG. 19;

FIGS. 22A and 22B are explanatory diagrams of Step S23 in FIG. 19;

FIGS. 23A and 23B are explanatory diagrams of Step S24 in FIG. 19; and

FIG. 24 is a flow chart showing part of the procedure of the method of treatment according to a modification of the second embodiment.

### DETAILED DESCRIPTION OF PREFERRED EMBODIMENTS

Hereinafter, each embodiment according to the present invention will be described with reference to the figures.

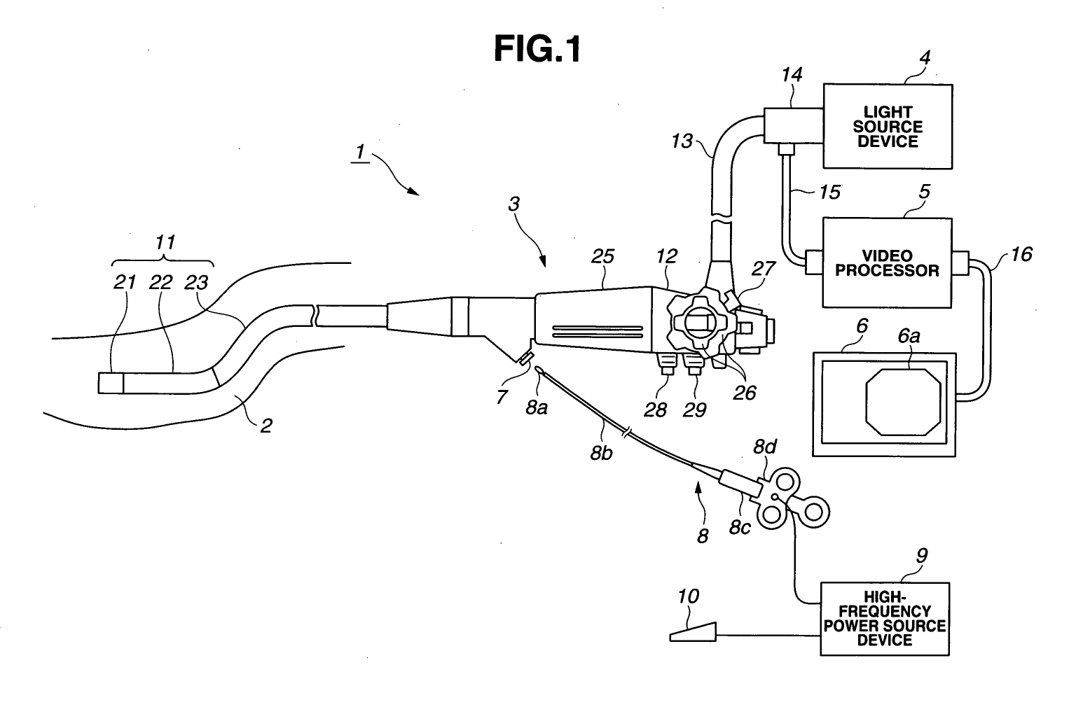

A first embodiment of the present invention is described with reference to FIGS. 1 to 9. FIG. 1 shows a configuration of an endoscope apparatus 1 according to the first embodiment of the present invention.

As shown in FIG. 1, the endoscope apparatus 1 has an endoscope 3 inserted in a body cavity 2, a light source device 4 for supplying an illumination light to the endoscope 3, a video processor 5 for performing signal processing for an image pickup device of the endoscope 3, and a monitor 6 as a display device which receives an input of a video signal from the video processor 5 to display an endoscope image corresponding to the video signal.

A high-frequency snare 8 as endo-therapy accessories used in a method of treatment according to this embodiment is insertable in an endo-therapy accessories insertion port 7 provided in the endoscope 3, while the high-frequency snare 8 is connected to a high-frequency power source device 9 via a cable.

The high-frequency power source device 9 is connected with a foot switch 10, for example, for turning on and off high-frequency current. By an operator turning on the foot switch 10, the high-frequency current is supplied from the high-frequency power source device 9 to the high-frequency snare 8 side.

The high-frequency current flows through a snare wire in a sheath portion 8b configuring the high-frequency snare 8 and into a site to be treated, such as an affected part, with which a snare loop portion 8a at the distal end of the snare wire is brought into contact, thereby excision procedure or the like of the site can be performed.

The high-frequency power source device 9 is connected with a counter electrode plate, which is not shown, forming a return path of the high-frequency current. The counter electrode plate is set so as to contact with buttocks or the like of a patient in a large area.

The high-frequency snare 8 has the elongated sheath portion 8b provided with the snare loop portion 8a as a loop shape portion at the distal end and a snare manipulation portion 8c used as manipulation means for varying a loop size of the snare loop portion 8a at the rear end.

The snare wire forming the snare loop portion 8a is inserted in the sheath portion 8b, and the rear end of the snare wire is connected to a slider 8d provided so as to be movable in the axial direction of the snare manipulation portion 8c.

By the operator manipulating the slider 8d to move to the rear side, for example, the snare wire can be towed to reduce the loop size of the snare loop portion 8a provided at the distal end of the sheath portion 8b.

The endoscope 3 has an elongated insertion portion 11 inserted in the body cavity 2 or the like, a manipulation portion 12 formed on the rear end of the insertion portion 11, and a universal cord 13 extended out from the manipulation portion 12. A light guide connector of a connector 14 at an end of the universal cord 13 is connected to the light source device 4, so that the illumination light is supplied from the light source device 4.

A cable 15 connected to the connector 14 is connected to the video processor 5. A cable 16 for transmitting the video signal outputted from the video processor 5 is connected to the monitor 6.

The insertion portion 11 has a rigid distal end forming portion (abbreviated as a distal end portion) 21 provided at the distal end thereof, a curvable bending portion 22 formed at the rear end of the distal end portion 21, and a flexible portion 23 having flexibility formed at the rear end of the bending portion 22. The proximal end side (rear end) of the flexible portion 23 is coupled to the manipulation portion 12.

The manipulation portion 12 includes a grip portion 25 provided at the distal end side of the manipulation portion 12 for being grasped by the operator, and a bending manipulation knob 26 for bending the bending portion 22 and a elevator control knob 27 for elevating an endo-therapy accessories forceps elevator 45, which will be described later, both provided at the rear end side of the manipulation portion 12.

On a side of the manipulation portion 12 adjacent to the side on which the bending manipulation knob 26 and the elevator control knob 27 are provided, there are provided an air/water feeding button 28 for feeding air/water and a suction button 29 for performing suction manipulation.

On the rear end of the manipulation portion 12, there is provided a scope switch for generating various instruction signals, such as a freeze instruction, for the video processor 5.

In the insertion portion 11, a light guide 31 (see FIGS. 3 and 4) for transmitting the illumination light supplied from the light source device 4 is inserted. The light guide 31 is bifurcated, for example, to be inserted in the insertion portion 11.

As shown in FIGS. 3 and 4, the distal end of the light guide 31 is fixed to the rear end side of a through hole forming an illumination window provided in a rigid distal end member 32 which configures the distal end portion 21.

An illumination optical system 33 is fixed in the front side of the through hole so as to oppose to the distal end surface of the light guide 31, and the illumination optical system 33 diffuses and emits the illumination light emitted from the distal end surface of the light guide 31 in the forward direction. A distal end cover 32a is attached to an outer peripheral surface at the distal end side of the distal end member 32.

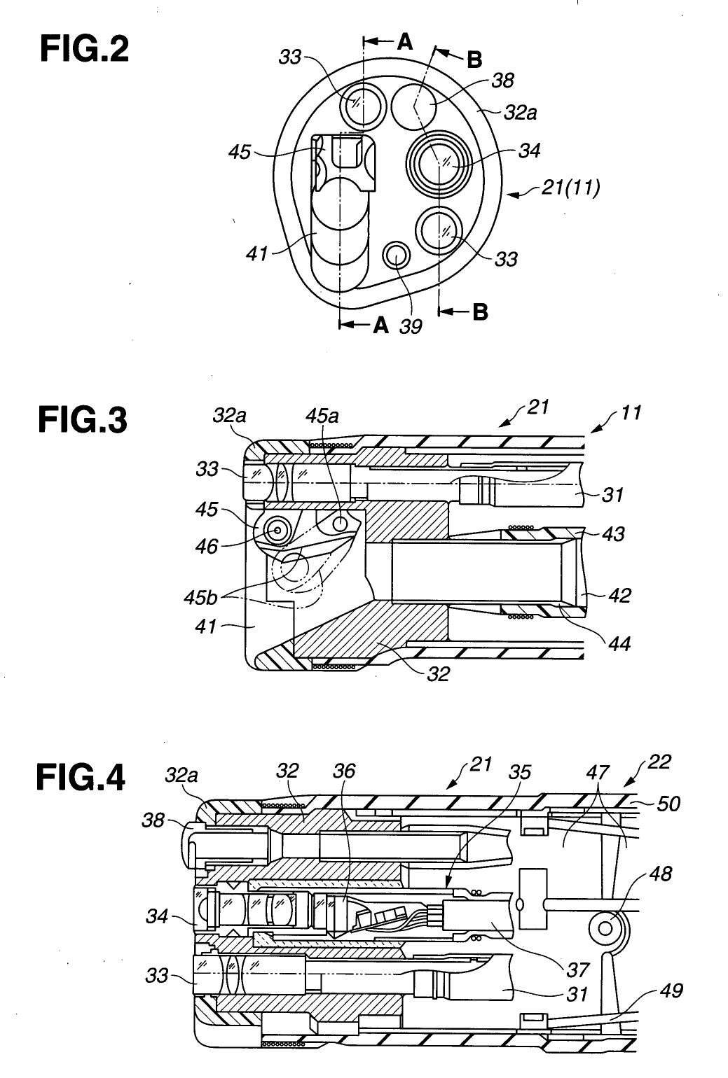

As shown in FIGS. 2 and 4, a through hole forming an observation window is provided adjacent to the illumination optical system 33 in the distal end portion 21, and an image pickup device 35 provided with an objective optical system 34 as an observation optical system is attached in the through hole.

In other words, the endoscope 3 is a direct view type endoscope recognizing the distal end side in the axial direction of the insertion portion 11 as an observation visual field of the objective optical system 34 (or the image pickup device 35), provided with illumination means for illuminating the observation visual field.

A charge coupled device (abbreviated as the CCD) 36, for example, as a solid-state image pickup sensor is arranged on an image forming position of the objective optical system 34 configuring the image pickup device 35, and photoelectrically converts an optical image formed in the CCD 36. The endoscope 3 is an electronic endoscope provided with the solid-state image pickup sensor.

A circuit board carrying a buffer amplifier or the like is provided on the rear side of the CCD 36, the circuit board being connected with the distal end of a cable 37.

The cable 37 is inserted in the insertion portion 11 or the like, and is connected with the video processor 5 through the connector 14 and the cable 15.

The video processor 5 includes an image processing circuit which performs signal processing for an output signal of the CCD 36 and generates the video signal.

The monitor 6 into which the video signal is inputted displays the image within the observation visual field picked up by the CCD 36 as an endoscope image 6a in an endoscope display area on a display surface of the monitor 6.

As shown in FIGS. 2 and 4, an air/water feeding nozzle 38 is provided adjacent to the objective optical system 34 of the observation window.

The operator can manipulate the air/water feeding button 28 to feed air or water from an opening at the distal end of the air/water feeding nozzle 38 to an outer surface of the opposing objective optical system 34, so as to set the outer surface of the objective optical system 34 to a clean state. As shown in FIG. 2, a front water feeding port 39 for feeding water forward is also provided.

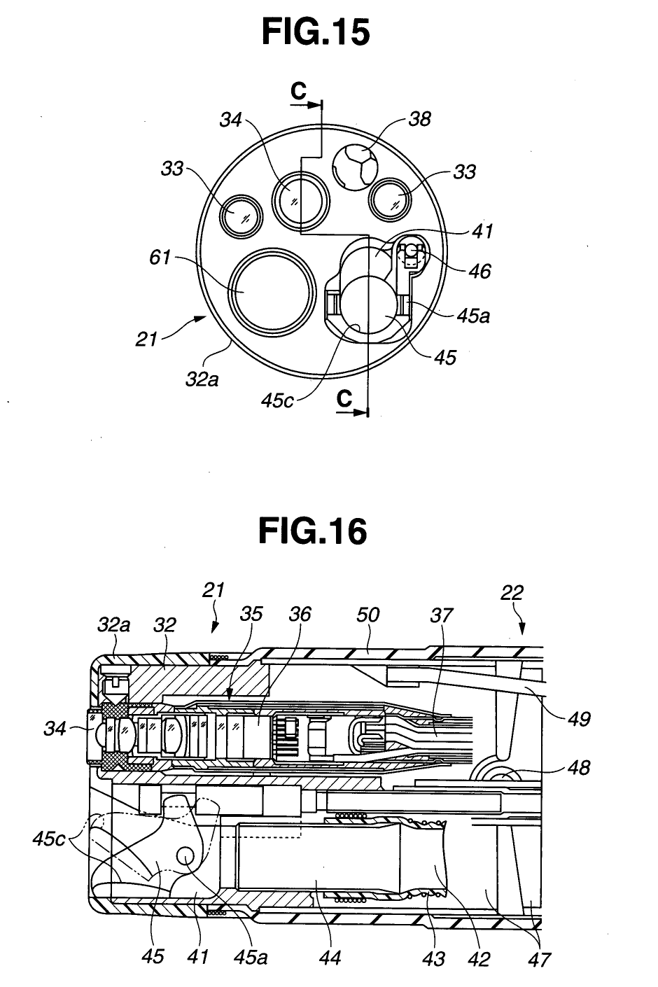

In FIGS. 2 through 4, the upper direction of the paper represents the upper direction when bending the bending portion 22 (the same is applicable to FIGS. 15, 16, 17, and 18, which will be described later).

The endoscope image 6a displayed on the monitor 6 is also displayed in the same manner that the upper direction when being displayed represents the upper direction.

As shown in FIG. 2, an opening 41 is provided adjacent to the right side of the observation window in the distal end portion 21.

As shown in FIG. 3, the opening 41 is a channel opening which communicates with the distal end of an endo-therapy accessories insertion channel (hereinbelow, abbreviated as the channel except for a case being ambiguous) 42 provided in the insertion portion 11 in the longitudinal direction.

The channel 42 provided in the insertion portion 11 is made of a flexible tube 43, with the distal end thereof being fixed to the rear end of a through hole forming the opening 41 above in the distal end member 32 via a pipe sleeve 44. The through hole has a widened front side which forms the opening 41 with the endo-therapy accessories forceps elevator (hereinafter, abbreviated simply as the forceps elevator) 45 for changing the projection direction of the endo-therapy accessories arranged therein.

More specifically, the forceps elevator 45 is arranged on the upper side, widened in a stepwise manner, inside the opening 41 so as to be rotatable about a shaft 45a provided at the rear end side thereof.

The distal end of a wire 46 is fixed to the front side of the shaft 45a of the forceps elevator 45. The wire 46 has the rear end coupled to a rotating member, for example, in the manipulation portion 12 through the insertion portion 11, so that the wire 46 can be towed by performing rotating manipulation of the elevator control knob 27.

A bottom surface 45b of the forceps elevator 45 is formed into a concave to function as a pressing portion. When the elevator control knob 27 is not rotated, the bottom surface 45b is substantially parallel to the longitudinal direction of the insertion portion 11, as shown by the solid line in FIG. 3.

In this state, the endo-therapy accessories inserted in the channel 42 are projected forward over the distal end of the opening 41 with hardly interfered by the bottom surface 45b.

In contrast, when the elevator control knob 27 is rotated, the forceps elevator 45 rotates about the shaft 45a, as shown by the dashed line in FIG. 3. By this rotation, the bottom surface 45b is rotatably moved so that the distal end side thereof enters downward into an insertion path of the endo-therapy accessories from above, which is the front side of the channel 42.

For this reason, if the endo-therapy accessories is inserted as described above, the endo-therapy accessories is pressed to move downward by the bottom surface 45b.

In other words, when the elevator control knob 27 is rotatably manipulated while the endo-therapy accessories is inserted below the bottom surface 45b, the projection direction of the distal end side of the endo-therapy accessories projected from the opening 41 is pressed to move downward, changing the projection direction of the distal end side of the endo-therapy accessories.

Since the projection direction of the endo-therapy accessories is changed from that before the rotating manipulation by rotatably manipulating the elevator control knob 27, this rotating manipulation is also called as elevator control.

The bottom surface side inside the opening 41 is widened downward, so that the endo-therapy accessories projected from the opening 41 can be variably projected within a range from the parallel direction to the obliquely downward direction with respect to the longitudinal direction of the distal end portion 21.

As shown in FIG. 4, a distalmost bending piece 47 which configures the bending portion 22 is fixed to the outer peripheral surface at the rear end of the distal end member 32. To the rear end of the bending piece 47, another bending piece 47 is rotatably coupled via a rivet 48. In this manner, the bending pieces 47 adjacent to each other in the longitudinal direction are rotatably coupled to form the bending portion 22.

In addition, tips of bending wires 49 are each fixed to the upper, lower, left, and right sides of the inner peripheral surface of the distalmost bending piece 47.

The rear end of the bending wire 49 is fixed to a rotating member, which is not shown, in the manipulation portion 12, and allows the bending portion 22 to bend in any of the upper, lower, left, and right directions by rotatably manipulating the bending manipulation knob 26.

The operator can manipulate the bending manipulation knob 26 to change the posture of the distal end portion 21 at the distal end of the bending portion 22 or shift observation visual field.

The outer peripheral surface of the bending piece 47 is covered with a casing tube 50.

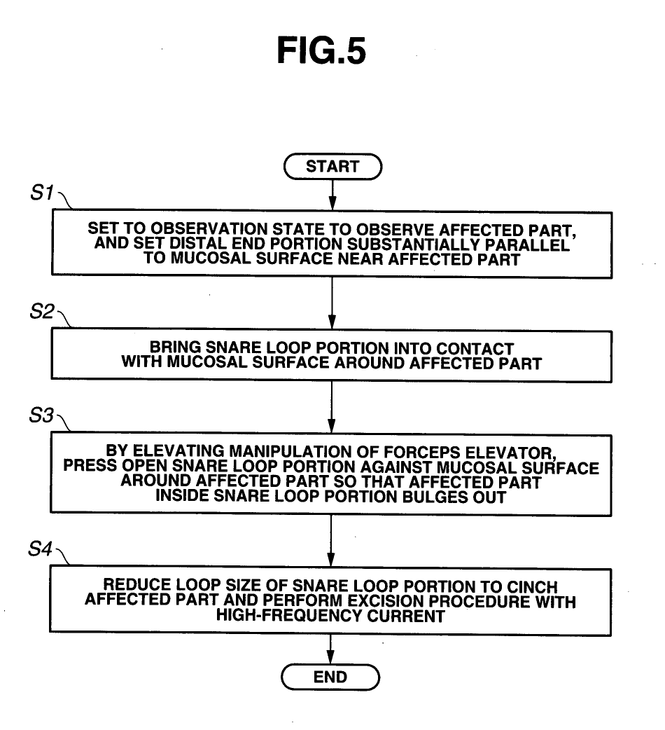

There is described below, with reference to FIG. 5, a method of treatment for the affected part that needs to be treated with the direct view type endoscope 3 having the configuration described above, by inserting the insertion portion 11 thereof in the body cavity 2.

For example, as shown in FIG. 1, the endoscope 3 is connected to the light source device 4 or the like, and the insertion portion 11 of the endoscope 3 is inserted in the body cavity 2 of the patient. Upon insertion, the operator pushes the rear end side of the insertion portion 11 forward so that the insertion portion 11 is inserted deep in the body cavity 2. In this case, the operator performs inserting operation while observing the endoscope image 6a displayed on the monitor 6.

The operator then manipulates the rear end side of the insertion portion 11 to control the posture of the distal end portion 21 so as to establish an observation state where a target of treatment in the body cavity 2, e.g., a slightly bulging affected part 51, is captured substantially near the center within the observation visual field of the objective optical system 34.

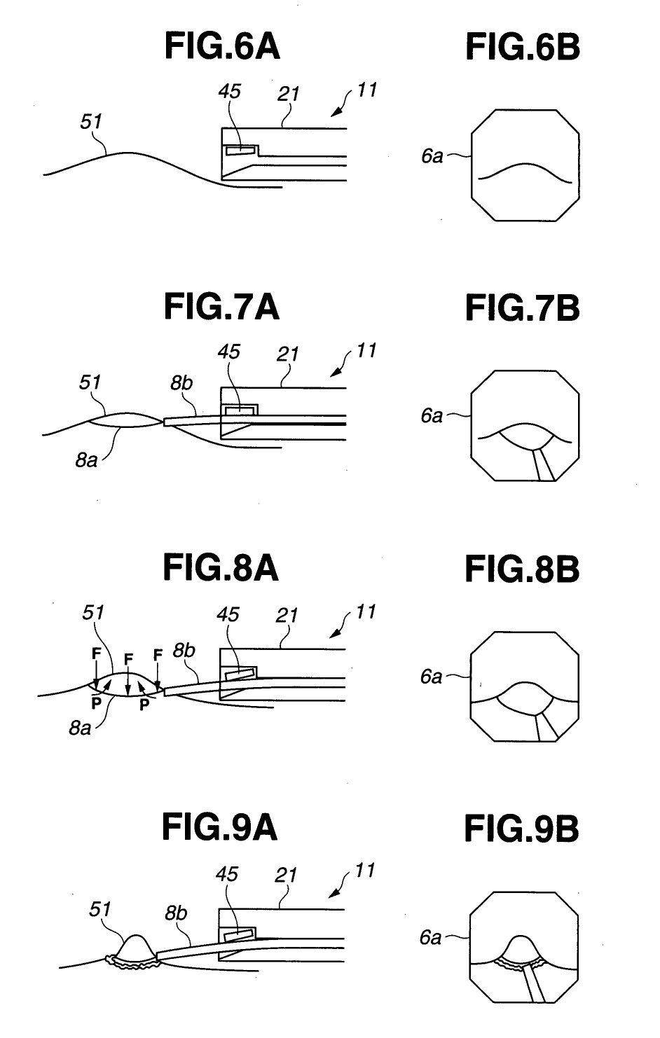

The operator also sets to the state where the distal end portion 21 side is substantially parallel to a mucosal surface near or around the affected part 51, as shown in FIG. 6A. The endoscope image 6a of the observation visual field obtained in this case would be as shown in FIG. 6B. FIG. 6A schematically shows the configuration near the distal end portion 21 of the insertion portion 11.

In other words, as shown in Step S1 of FIG. 5, the operator sets (the distal end portion 21 side) to the observation state where the affected part 51 can be observed within the observation visual field, and establishes the state where the distal end portion 21 is substantially parallel to the mucosal surface near or around the affected part 51.

Next, the operator inserts the high-frequency snare 8 as the endo-therapy accessories from the snare loop portion 8a side of the distal end portion thereof in the endo-therapy accessories insertion port 7, to cause the distal end side of the high-frequency snare 8 to project from the front side of the opening 41 at the distal end of the channel 42.

Thereafter, as shown in Step S2 of FIG. 5, the operator brings the substantially circular snare loop portion 8a projected from the opening 41 into contact with the mucosal surface around the affected part 51 so as to surround it. In other words, the state as shown in FIGS. 7A and 7B is established. The operator brings the loop into contact with the mucosal surface while the loop is widened, as shown in FIG. 7B.

In this case, it is desirable that the whole circular loop of the snare loop portion 8a is in contact with the mucosal surface.

Here, as shown in FIG. 7B, the distal end side of the high-frequency snare 8 is projected in the direction substantially parallel to the longitudinal direction of the distal end portion 21. The forceps elevator 45 in this state is not elevated.

In this state, the operator establishes the contact with the mucosal surface so that a plane including the loop of the snare loop portion 8a (referred to as a loop plane) is substantially orthogonal to a predetermined direction (specifically, from the upward to the downward direction) of the forceps elevator 45 to be elevated. When considering an aspect of the predetermined direction, the operator sets the loop plane to be in contact with the upper side of the mucosal surface. In this case, the loop plane is set to be substantially horizontal in the observation visual field.

The operator then rotates the elevator control knob 27 provided on the manipulation portion 12 to perform the elevator control for elevating the forceps elevator 45.

With this elevator control, a elevating wire is towed and the forceps elevator 45 is rotated about the shaft 45a, so that the forceps elevator 45 in the state where the endo-therapy accessories projects over it in substantially horizontal thereto is rotated (elevated) in the lower direction.

As the forceps elevator is elevated, a pressing surface thereof presses (biases) the sheath portion 8b of the high-frequency snare 8 downward from above.

A distal end side portion of the high-frequency snare 8 projected over a pressed part is moved downward as for its direction projected from the opening 41.

With this movement, the snare loop portion 8a presses the mucosal surface downward. FIG. 8A shows this state. As shown in FIG. 8A, the snare loop portion 8a presses the mucosal surface downward with a press force represented by reference character F. As pressed in this manner, the affected part 51 portion inside the snare loop portion 8a bulges out upward as shown by reference character P.

In other words, as in Step S3 of FIG. 5, the snare loop portion 8a presses the mucosal surface around the affected part 51 by the elevator control of the forceps elevator 45, causing the affected part 51 to bulge out with the press force F.

FIGS. 8A and 8B show the state where the affected part has bulged out as described above. As shown in FIG. 8B, the affected part 51 inside the snare loop portion 8a can be bulged out while maintaining the observation visual field substantially similar to the observation visual field of FIG. 7B. Complementarily, since the elevator control is performed in the state of the observation visual field of FIG. 7B, this elevator control can avoid moving the distal end portion 21.

In contrast, although the affected part 51 can be bulged out by bending the bending portion 22 downward in the state of the observation visual field of FIG. 7B, the observation visual field is shifted from the state of FIG. 7B at the time. Therefore, the observation visual field is shifted even if it has been set to be convenient for observation as in FIG. 7B.

The operator then performs treatment for the affected part 51, as shown in Step S4. Specifically, the operator manipulates the snare manipulation portion 8c to reduce the loop size of the snare loop portion 8a and cinch the bulging affected part 51, and then sends the high-frequency current through the snare loop portion 8a by turning on the foot switch 10, thereby performing the excision procedure of the affected part 51 with the high-frequency current.

With this procedure, the affected part 51 can be excised as shown in FIGS. 9A and 9B.

Such a method of treatment has an advantage to allow for performing the effective treatment with simple manipulation in such a case that an early lesion with a small bulge is excised.

The second prior art document cited above discloses a method of treatment of the affected part 51 related to the method of treatment herein. However, the second prior art document does not suggest that the affected part 51 is bulged out by elevating the forceps elevator as in the method of treatment herein.

In this embodiment, during Step S1 through Step S4 of FIG. 5, the excision procedure of the affected part 51 using the snare loop portion 8a can be smoothly performed while maintaining the observation state where the affected part 51 is substantially placed near the center of the observation visual field.

Particularly, since the operator performs the treatment of Step S2 through Step S3 by elevating the forceps elevator 45, the state of the observation visual field for observing the affected part 51 is not altered. In contrast, although the affected part 51 can also be bulged out by bending the bending portion 22, for example, as described above, it causes the direction of the observation visual field to shift, which may result in an inconvenient observation state where the bulging part is shifted away from the observation visual field or shifted to an edge of the observation visual field.

Next, a modification of the first embodiment is described. In the first embodiment, there has been described the method of performing the treatment by setting the distal end side of the insertion portion 11 in substantially parallel to the mucosal surface around the affected part 51.

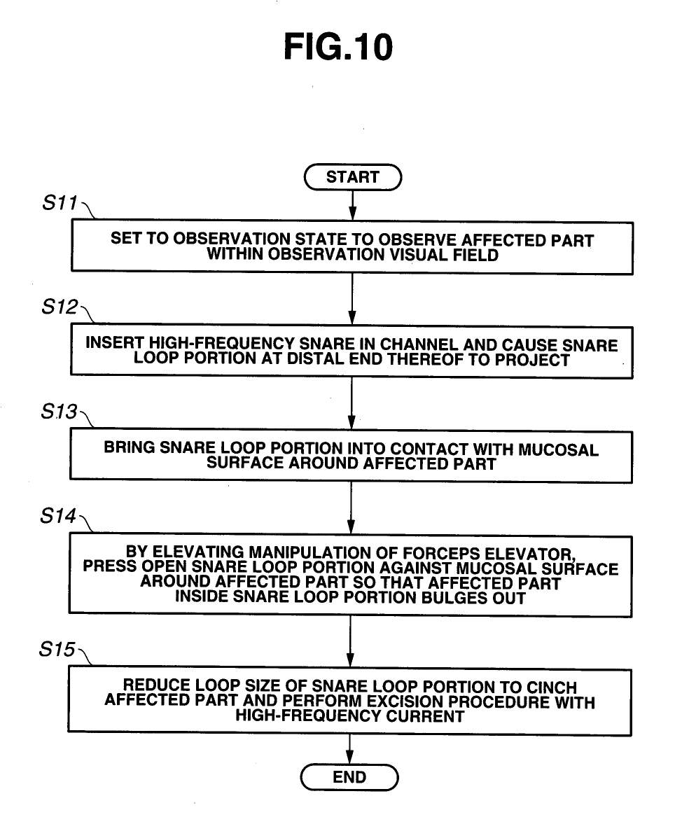

In contrast, this modification corresponds to the case where the treatment is performed by setting the distal end side of the insertion portion 11 to be angled, or inclined, with respect to the mucosal surface. FIG. 10 shows a flow chart of the method of treatment of this modification.

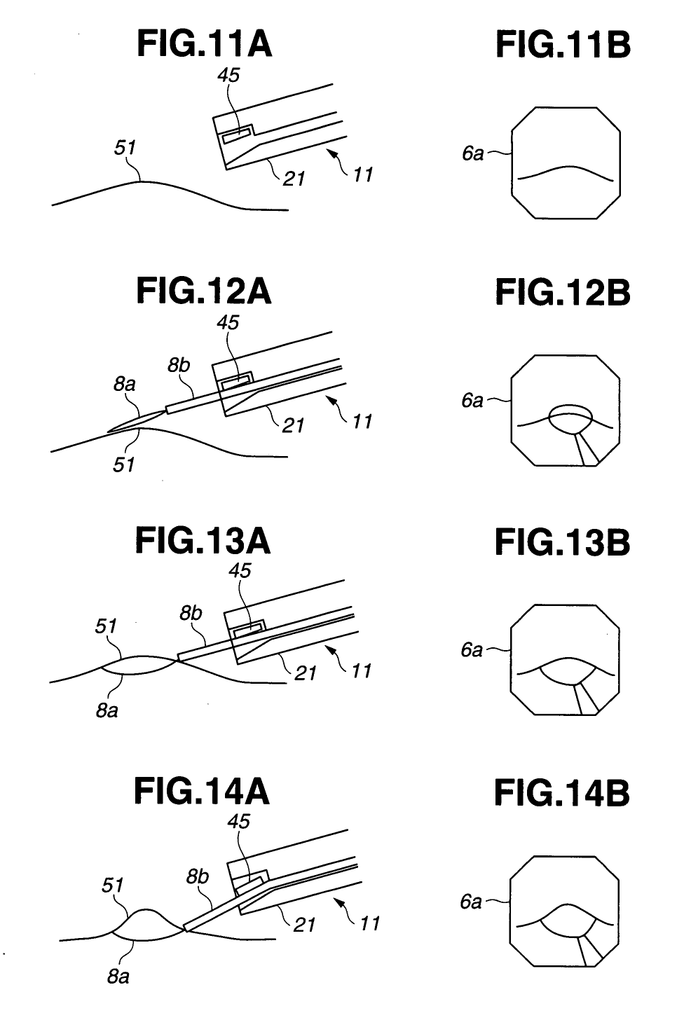

In the first step S11, the operator sets the distal end portion 21 to establish the observation state to observe the affected part 51 substantially near the center of the observation visual field, as shown in FIGS. 11A and 11B.

In this observation state, the distal end portion 21 is set to be inclined with respect to the mucosal surface instead of the state in FIG. 6A where the distal end portion 21 is substantially parallel to the mucosal surface.

Next, the operator inserts the high-frequency snare 8 as the endo-therapy accessories from the snare loop portion 8a side of the distal end portion thereof in the endo-therapy accessories insertion port 7, as shown in Step S12 of FIG. 10, to project the distal end side of the high-frequency snare 8 forward in the opening 41 at the distal end of the channel 42.

In other words, the state as shown in FIGS. 12A and 12B is established. Then, the substantially circular shaped loop of the projected snare loop portion 8a is widened as shown in FIG. 12B.

Thereafter, as shown in Step S13 of FIG. 10, the operator brings the substantially circular snare loop portion 8a projected from the opening 41 into contact with the mucosal surface around the affected part 51 so as to surround it. In order to establish the contact, the posture of the distal end portion 21 is controlled if needed. The state as shown in FIGS. 13A and 13B is established.

In this states the loop plane of the snare loop portion 8a is in contact with the mucosal surface downward from above.

The operator then rotates the elevator control knob 27 provided on the manipulation portion 12 to perform the elevator control for elevating the forceps elevator. With this elevator control, the elevating wire is towed and the forceps elevator 45 is rotated about the shaft 45a, so that the forceps elevator 45 in the state where the pressing surface thereof is substantially parallel to the longitudinal direction of the insertion portion 11 is rotated (elevated) in the lower direction.

As the forceps elevator 45 is elevated, the pressing surface thereof presses (biases) the sheath portion 8b of the high-frequency snare 8 downward. The distal end side portion of the high-frequency snare 8 projected over the pressed part is moved downward as for its direction projected from the opening 41.

With this movement, the snare loop portion 8a presses the mucosal surface downward. As pressed in this manner, the affected part 51 portion inside the snare loop portion 8a bulges out upward.

In other words, as in Step S14 of FIG. 10, the snare loop portion 8a is pressed against the mucosal surface of the affected part 51 by the elevator control for the forceps elevator 45, causing the affected part 51 to bulge out with the press force.

FIGS. 14A and 14B show this state. As shown in FIG. 14B, the affected part 51 inside the snare loop portion 8a can be bulged out while maintaining the observation visual field substantially similar to the observation visual field of FIG. 13B.

The operator then performs the treatment for the affected part 51, as shown in Step S15. Specifically, the operator manipulates the snare manipulation portion 8c to reduce the loop size of the snare loop portion 8a and cinch the bulging affected part 51, and then turns on the foot switch 10, thereby performing the excision procedure of the affected part 51. This procedure is similar to that in Step S4 of FIG. 5.

Such a method of treatment has the advantage, as with the first embodiment, to allow for performing the effective treatment with simple manipulation in such a case that an early lesion with a small bulge is excised.

The method of treatment above has been described as it adopts the endoscope 3 having a structure that the projection direction of the distal end side of the endo-therapy accessories inserted in the channel 42 is moved downward when the elevator control knob 27 is rotatably manipulated.

It is not limited to the endoscope 3 having such a structure but a modified endoscope as shown in FIGS. 15 and 16, for example, may be adopted. FIGS. 15 and 16 show a front view of the distal end portion 21 side of the endoscope according to the modification and a C-C sectional view thereof, respectively.

Also in FIGS. 15 and 16, the upper direction of the paper represents the upper direction when bending the bending portion 22. As shown in FIG. 15, the image pickup device 35 (see FIG. 16) provided with the objective optical system 34 is arranged slightly above a position corresponding to the center of the distal end surface of the distal end portion 21.

Two illumination optical systems 33 and 33 are arranged on left and right sides of the objective optical system 34, and the air/water feeding nozzle 38 is arranged at a position obliquely above the objective optical system 34.

In addition, the opening 41, which is in communication with the channel 42 and includes the forceps elevator 45 arranged therein, and a suction channel 61 are arranged below the objective optical system 34.

As for the endoscope 3 shown in FIGS. 1 through 4, the channel 42 inserted in the insertion portion 11 is branched into the endo-therapy accessories insertion port 7 side and the suction channel extended out toward the manipulation portion 12 side near the rear end of the insertion portion 11. In other words, in this endoscope 3, the channel 42 is used both as the endo-therapy accessories insertion channel and the suction channel in the insertion portion 11.

In contrast, in the endoscope shown in FIGS. 15 and 16, the (endo-therapy accessories insertion) channel 42 and the suction channel 61 are independently provided in the insertion portion.

As shown in FIG. 16, the forceps elevator 45 arranged in the opening 41 has the upper surface (inner surface) 45c in the bottom serving as the pressing surface, which guides the endo-therapy accessories forward, as shown by the solid line, when not elevated. When elevated, the forceps elevator 45 moves the projection direction of the endo-therapy accessories upward, as shown by the two-dot chain line. Other components similar to those described above in the first embodiment are given the same reference numerals, and the description thereof is omitted.

The method of treatment described above is similarly applicable to the case where the endoscope shown in FIGS. 15 and 16 is used. However, in this case, the endoscope image 6a observed on the monitor 6 can be set to be an inverted image shown in FIGS. 6B through 9B or FIGS. 11B through 14B. This is because, as for the endoscope shown in FIGS. 15 and 16, the elevator control moves the endo-therapy accessories in the opposite direction as compared with that when the endoscope 3 shown in the figures including FIG. 3 is used.

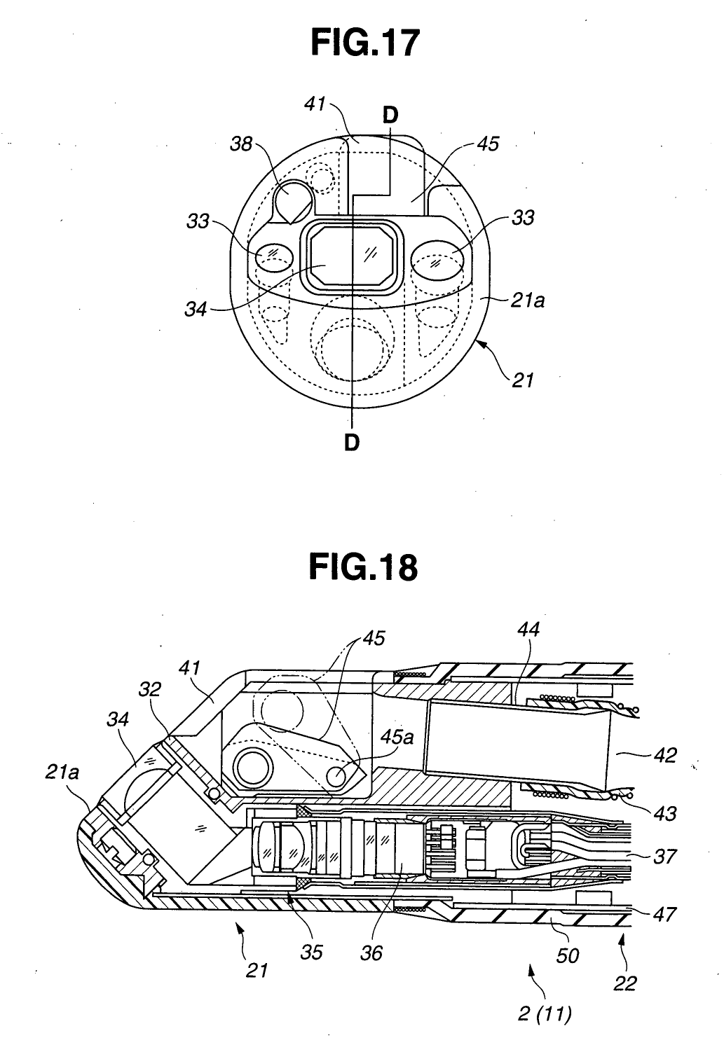

Next, a second embodiment of the present invention is described with reference to FIGS. 17 through 23. FIGS. 17 and 18 show a configuration near the distal end portion 21 of an endoscope 3C according to the second embodiment of the present invention. The endoscope 3C according to this embodiment is an oblique view type electronic endoscope.

As shown in FIGS. 17 and 18, the distal end portion 21 of the insertion portion 11 forms a distal end surface 21a inclined in the obliquely upward direction by a degree greater than 0 degree with respect to the longitudinal direction of the distal end portion 21. The image pickup device 35 provided with the objective optical system 34 in the direction of an oblique visual field is provided near the center of the distal end surface 21a. The illumination optical systems 33 are arranged on the both sides of the objective optical system 34. In addition, the opening 41 in communication with the channel 42 is provided above the objective optical system 34, in which the forceps elevator 45 is arranged so as to be rotatable about the shaft 45a.

The forceps elevator 45 rotates from a position shown by the solid line to that shown by the two-dot chain line when rotated about the shaft 45a, which causes the upper surface of the forceps elevator 45 to press the endo-therapy accessories inserted in the channel 42 from beneath to move it upward.

In addition, the air/water feeding nozzle 38 is arranged obliquely above the objective optical system 34. Other components similar to those in the first embodiment are given the same reference numerals, and the description thereof is omitted.

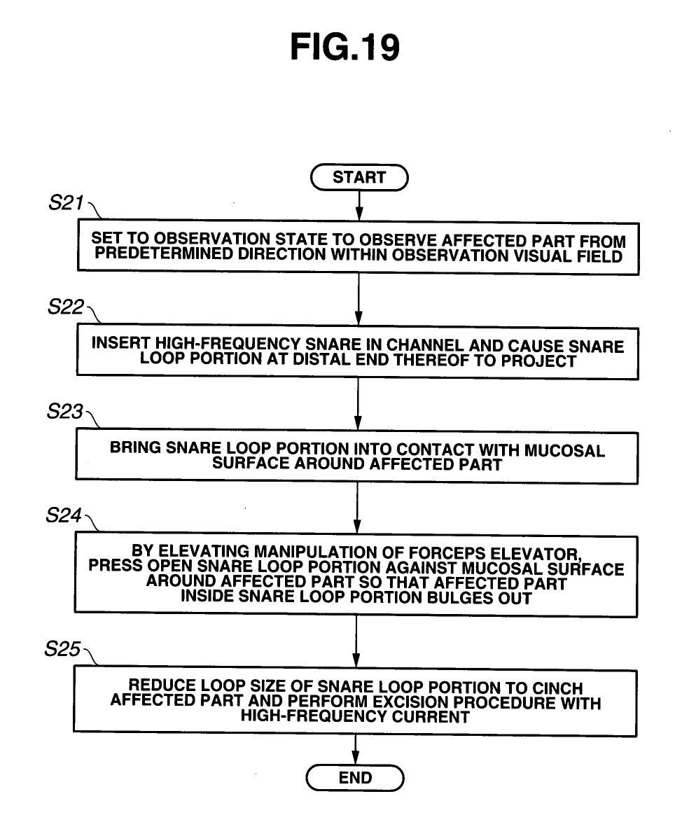

Next, the method of treatment according to the second embodiment of the present invention is described with reference to a flow chart of FIG. 19.

The method of treatment shown in FIG. 19 is fundamentally similar to that of the first embodiment or its modification.

In the first Step S21, the operator sets to the observation state to observe the affected part 51 from a predetermined direction within the observation visual field. Here, the predetermined direction corresponds to the moving direction of the endo-therapy accessories when the forceps elevator 45 is elevated. In this embodiment, it corresponds to the upward direction.

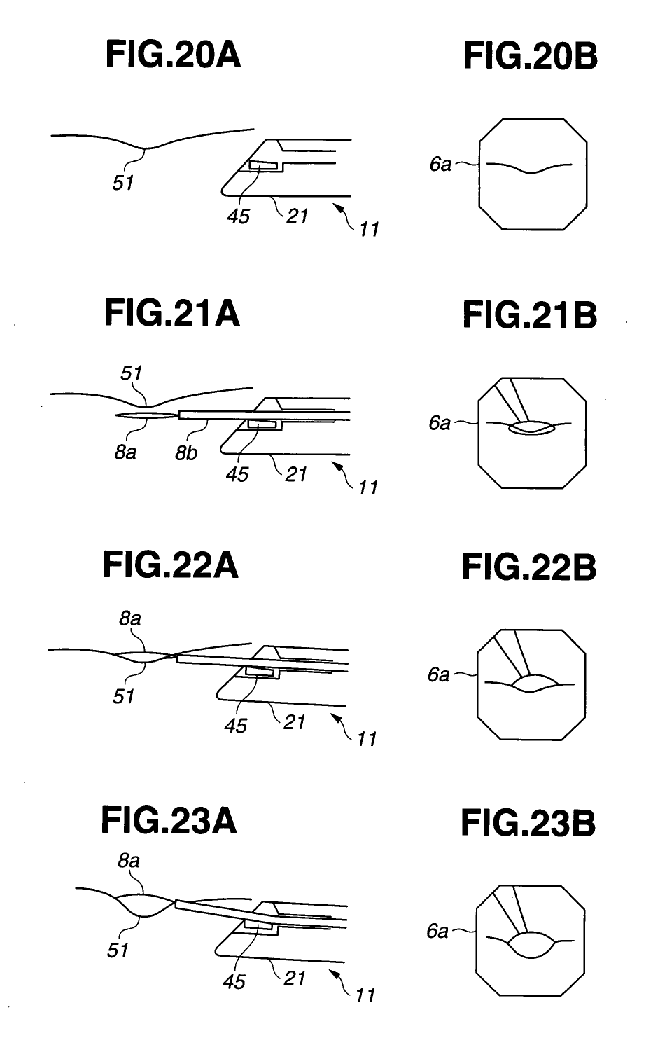

FIG. 20A shows an aspect of the distal end side of the insertion portion 11 in this state, and FIG. 20B shows the endoscope image 6a at the time.

As shown in Step S22, the operator then inserts the high-frequency snare 8 in the channel 42 to cause the snare loop portion 8a at the distal end thereof to project from the distal end of the opening 41. In addition, the loop shape of the projected snare loop portion 8a is widened. FIG. 21A shows this state and FIG. 21B shows the endoscope image 6a at the time.

Then, as shown in Step S23, the operator brings the projected snare loop portion 8a into contact with the mucosal surface near or around the affected part 51. In order to achieve it, the operator controls the posture of the distal end portion 21 side to slightly move from the state of FIG. 21A toward the mucosal surface side, for example. In this case, the operator may bring the snare loop portion 8a into contact with the mucosal surface near or around the affected part 51 by bending the bending portion 22 slightly upward.

FIG. 22A shows this state and FIG. 22B shows the endoscope image 6a at the time.

Next, as shown in Step S24, the operator performs the elevator control to elevate the forceps elevator 45 by rotating the elevator control knob 27 provided on the manipulation portion 12 (of FIG. 1). With this elevator control, the elevating wire is towed and the forceps elevator 45 is rotated about the shaft 45a, so that the forceps elevator 45 in the state where the pressing surface thereof is substantially parallel to the longitudinal direction of the insertion portion 11 is rotated (elevated) in the upper direction.

As the forceps elevator 45 is elevated, the pressing surface thereof presses (biases) the sheath portion 8b of the high-frequency snare 8 upward. The distal end side portion of the high-frequency snare 8 projected over the pressed part is moved upward as for its direction projected from the opening 41.

With this movement, the snare loop portion 8a presses the mucosal surface upward. As pressed in this manner, the affected part 51 portion inside the snare loop portion 8a bulges out downward.

FIGS. 23A and 23B show this state. As shown in FIG. 23B, the affected part 51 inside the snare loop portion 8a can be bulged out while maintaining the observation visual field substantially similar to the observation visual field of FIG. 22B.

The operator then performs the treatment for the affected part 51, as shown in Step S25. Specifically, the operator manipulates the snare manipulation portion 8c to reduce the loop size of the snare loop portion 8a and cinch the bulging affected part 51, and then turns on the foot switch 10, thereby performing the excision procedure of the affected part 51.

Such a method of treatment has an advantage, as with the first embodiment, to allow for performing the effective treatment with simple manipulation in such a case that an early lesion with a small bulge is excised.

The method of treatment shown in FIG. 19 may be performed with the distal end portion 21 side being set substantially parallel to the mucosal surface, as described in the first embodiment, or set inclined with respect to the mucosal surface.

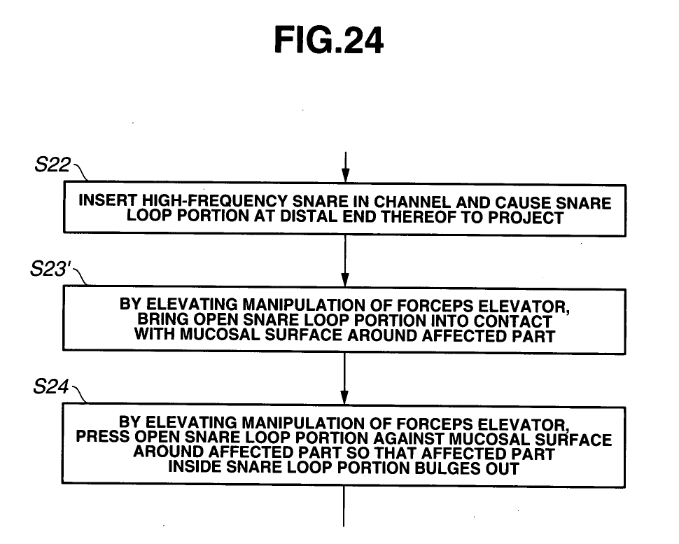

Aside from the method of treatment described above in the second embodiment, a modified method of treatment as shown in FIG. 24 may be adopted. This method of treatment is achieved by changing Step S23 of FIG. 19 as follows, for example.

For example, when Step S22 of FIG. 19 is performed, the state of the observation visual field is set, for example, as that of FIG. 21B. Assume that the snare loop portion 8a is close to and opposed to the affected part 51, as shown in FIG. 21B.

In this case, with the elevator control for the forceps elevator 45 as shown in Step S23′ of FIG. 24 while maintaining the state of the observation visual field, the snare loop portion 8a is brought into contact with the mucosal surface near or around the affected part 51.

In other words, if the snare loop portion 8a is close to and opposed to the affected part 51 as shown in FIG. 21B, the snare loop portion 8a can be brought into contact with the surface near the affected part 51 by slightly elevating the forceps elevator 45.

Thereafter, as shown in Step S24 of FIG. 19, the operator further performs the elevator control to elevate the forceps elevator 45.

When the snare loop portion 8a is close to and opposed to the affected part 51 as shown in FIG. 21B, this modification allows the operator to readily bring the snare loop portion 8a into contact with the surface of the affected part 51 by a small manipulation amount of the elevator control. Furthermore, the operator can cause the affected part 51 to bulge out by a larger manipulation amount of the elevator control.

In this modification as well, a locked state is set where the observation visual field is hardly shifted and the affected part bulges out, thereby the excision procedure of the affected part 51 can be smoothly performed.

When bringing the snare loop portion 8a into contact with the surface near or around the affected part 51 as described above, it is desirable that the loop plane contacts with the surface from the moving direction of the distal end portion of the endo-therapy accessories by the elevator control of the forceps elevator 45.

Since many forceps elevators 45 have the structure that it moves the distal end side of the endo-therapy accessories upward or downward by the elevator control, the loop plane may be set to be observed near the center of the observation visual field while being set in the substantially horizontal direction (transverse direction as defined based on the bending direction of the bending portion 22) when bringing the snare loop portion 8a into contact with the surface near or around the affected part 51.

Each of the embodiments or the like described above has been described with regard to the electronic endoscope, such as the endoscope 3 provided with the image pickup device 35. The present invention is not limited to the method of treatment performed using the electronic endoscope provided with the image pickup device 35, but is also applicable to the case where the treatment is performed using an optical endoscope, such as a fiberscope.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A method of treatment with an endoscope for treating an affected part to be treated, using the endoscope having an observation optical system and an opening, opened to the distal end of an endo-therapy accessories insertion channel, at a distal end portion of an insertion portion provided with the endo-therapy accessories insertion channel therein, and having an endo-therapy accessories forceps elevator arranged in the opening for elevating endo-therapy accessories in a predetermined direction, the endo-therapy accessories including a loop shape portion projected from the opening at the distal end of a sheath portion, comprising:
a first step of setting to an observation state to observe the affected part within an observation visual field by the observation optical system and bringing the loop shape portion projected from the opening, in a state that a loop of the loop shape portion is widened, into contact with a surface near the affected part; and
a second step of pressing the widened loop shape portion against the surface near the affected part by elevator control for elevating the endo-therapy accessories forceps elevator while maintaining the state where the loop shape portion is in contact with the surface near the affected part, so as to cause the affected part inside the loop shape portion to bulge out.

2. The method of treatment with the endoscope according to claim 1, comprising a third step of setting the distal end side of the insertion portion to be substantially parallel to the surface near the affected part before bringing the loop shape portion into contact with the surface near the affected part.

3. The method of treatment with the endoscope according to claim 1, comprising a fourth step of treating the bulging affected part after the second step.

4. The method of treatment with the endoscope according to claim 3, wherein the fourth step is of treating the affected part by reducing a loop size of the loop shape portion and applying high-frequency current to the loop shape portion side.

5. The method of treatment with the endoscope according to claim 1, wherein a locked state, where the observation visual field by the observation optical system is not shifted, is maintainable when performing treatment of the second step after the first step.

6. The method of treatment with the endoscope according to claim 1, wherein the endoscope is of a direct view type where a direction of a visual field of the observation optical system is in a forward direction parallel to the longitudinal direction of the distal end portion of the insertion portion.

7. The method of treatment with the endoscope according to claim 1, wherein the endoscope is of an oblique view type where a direction of a visual field of the observation optical system is in an obliquely forward direction having an angle greater than zero degree with respect to the longitudinal direction of the distal end portion of the insertion portion.

8. The method of treatment with the endoscope according to claim 1, wherein, in the first step, the affected part is set to the state where it is observed near the center of the observation visual field.

9. The method of treatment with the endoscope according to claim 1, wherein the endoscope is an electronic endoscope in which a solid-state image pickup sensor is arranged on an image forming position of the observation optical system.

10. The method of treatment with the endoscope according to claim 1, wherein an image within a range of the visual field by the observation optical system is displayed on a display device via a signal processing device for performing signal processing for the solid-state image pickup sensor.

11. The method of treatment with the endoscope according to claim 1, wherein a proximal end side of the endo-therapy accessories is connected to a high-frequency power source device for supplying the high-frequency current to the loop shape portion.

12. The method of treatment with the endoscope according to claim 1, wherein the endo-therapy accessories includes a manipulation portion on the proximal end side of the endo-therapy accessories for changing a size of a loop shape of the loop shape portion.

13. The method of treatment with endoscope according to claim 1, wherein, in the endoscope includes a elevator control portion on the proximal end side of the insertion portion for elevating the endo-therapy accessories forceps elevator in a predetermined direction.

14. The method of treatment with the endoscope according to claim 1, wherein, in the first step, a plane including the loop shape portion is set in a direction substantially orthogonal to the predetermined direction in which the endo-therapy accessories is elevated when the endo-therapy accessories forceps elevator is elevated.

15. The method of treatment with the endoscope according to claim 1, wherein, in the first step, the plane including the loop shape portion is set in a substantially horizontal direction in the observation visual field.

16. A method of treatment with an endoscope for treating an affected part to be treated, using the endoscope having an observation optical system and an opening, opened to the distal end of an endo-therapy accessories insertion channel, at a distal end portion of an insertion portion provided with the endo-therapy accessories insertion channel therein, and having an endo-therapy accessories forceps elevator arranged in the opening for elevating endo-therapy accessories in a predetermined direction, the endo-therapy accessories including a loop shape portion projected from the opening at the distal end of a sheath portion, comprising:
a first step of setting to an observation state to observe the affected part within an observation visual field by the observation optical system while setting the distal end side to be substantially parallel to a surface near the affected part;
a second step of bringing the loop shape portion projected from the opening, in a state that a loop of the loop shape portion is widened, into contact with the surface near the affected part; and
a third step of pressing the widened loop shape portion against the surface near the affected part by elevator control for elevating the endo-therapy accessories forceps elevator while maintaining the state where the loop shape portion is in contact with the surface near the affected part, so as to cause the affected part inside the loop shape portion to bulge out.

17. The method of treatment with the endoscope according to claim 16, comprising a fourth step of treating the bulging affected part after the third step.

18. The method of treatment with the endoscope according to claim 17, wherein the fourth step is of treating the affected part by reducing a loop size of the loop shape portion and applying high-frequency current to the loop shape portion side.

19. The method of treatment with the endoscope according to claim 16, wherein a locked state, where the observation visual field by the observation optical system is not shifted, is maintainable when performing treatment of the third step after the second step.

20. The method of treatment with the endoscope according to claim 16, wherein, in the first step, the affected part is set to the state where it is observed near the center of the observation visual field.

21. The method of treatment with the endoscope according to claim 16, wherein, in the second step, a plane including the loop shape portion is set in a direction substantially orthogonal to the predetermined direction in which the endo-therapy accessories is elevated when the endo-therapy accessories forceps elevator is elevated.

22. A method of treatment with an endoscope for treating an affected part to be treated, using the endoscope having an observation optical system and an opening, opened to the distal end of an endo-therapy accessories insertion channel, at a distal end portion of an insertion portion provided with the endo-therapy accessories insertion channel therein, and having an endo-therapy accessories forceps elevator arranged in the opening for elevating endo-therapy accessories in predetermined direction, the endo-therapy accessories including a loop shape portion projected from the opening at the distal end of a sheath portion, comprising:
a step of setting to an observation state to observe the affected part within an observation visual field by the observation optical system, maintaining a locked state of an observation area by the observation optical system while the loop shape portion projected from the opening, in a state that a loop of the loop shape portion is widened, is in contact with a surface near the affected part, and pressing the loop shape portion against the surface of the affected part by elevator control for elevating the endo-therapy accessories forceps elevator so as to cause the affected part inside the loop shape portion to bulge out.
